(19) United States
(12) Patent Application Publication
(10) Pub. No.: US 2014/0199681 A1
(43) Pub. Date: Jul. 17, 2014

(54) BLOOD COLLECTION DEVICE FOR STABILIZING CELL-FREE RNA IN BLOOD DURING SAMPLE SHIPPING AND STORAGE

(71) Applicant: Streck, Inc., LaVista, NE (US)

(72) Inventors: Wayne L. Ryan, Omaha, NE (US); M. Rohan Fernando, Omaha, NE (US); Jianbing Qin, Omaha, NE (US)

(73) Assignee: Streck, Inc., LaVista, NE (US)

(21) Appl. No.: 14/153,204

(22) Filed: Jan. 13, 2014

Related U.S. Application Data
(60) Provisional application No. 61/751,983, filed on Jan. 14, 2013.

(51) Int. Cl. C12Q 1/68

(52) U.S. Cl. CPC C12Q 1/6806
USPC 435/2

(57) ABSTRACT

A method for preserving and protecting cell-free nucleic acids located within blood plasma samples is disclosed, wherein a sample of blood containing nucleic acids is treated to reduce deleterious effects of storage and transport.

### FIELD OF THE INVENTION

This invention relates to a device and method for improved protection and regulation of nucleic acid materials during collection, storage, and shipment.

### BACKGROUND OF THE INVENTION

Cell-free RNA (cfRNA) naturally occurs in blood and has the potential to be used for non-invasive prenatal diagnostics and for detection, monitoring, and molecular analysis of biomarkers for cancer and other diseases. Since cfRNA targets are present in blood at low quantities, it is important to minimize release of cellular RNA in a blood sample following blood draw. Pre-analytical conditions can affect the release of background (e.g., cellular) RNA into plasma, decreasing the proportion of specific cfRNA targets and masking their detection in downstream applications (e.g., polymerase chain reaction, flow cytometric and other analysis protocols). Due to the low abundance of the cfRNA biomarkers, it is recommended that genomic RNA background levels be minimized to provide accurate measurements cfRNA levels. Therefore, it is necessary to address several pre-analytical issues that arise during the time between blood draw and RNA isolation. These include delays in blood processing, blood storage temperature, and agitation of the sample during transport and shipment of blood. Such conditions may alter plasma RNA levels by causing cellular RNA release from blood cells that obfuscate true cfRNA. There is thus a clear need for protocols that stabilize cfRNA in blood as well as maintain cfRNA integrity during sample processing and shipping.

### SUMMARY OF THE INVENTION

The cell-free RNA collection devices disclosed herein prevent increases in background RNA levels caused by temperature fluctuations or agitation that can occur during blood sample storage and shipping. These blood collection devices provide a method for obtaining high quality stabilized cfRNA samples for rare RNA target detection and determining accurate cfRNA concentrations.

In one aspect, the present teachings contemplate a method including drawing a blood sample directly into a stabilizing blood collection device at a blood draw site, such blood collection device including an effective amount of a stabilizing agent selected from imidazolidinyl urea, diazolidinyl urea, aurintricarboxylic acid, glycine, glyceraldehyde, sodium fluoride or combinations thereof. The sample of blood includes an initial amount of background RNA and an amount of cell free RNA. The method may further include treating one or more components of the sample with the stabilizing agent for mitigating the propensity of background RNA within the sample to increase relative to the initial amount. The method may include a step of handling the sample, while it remains in the blood collection device, for delivery to a site at which cell-free RNA analysis is to be performed, during which transporting step the blood collection device and the sample contained therein is subjected to one or more temperatures within a range of about 5 to about 35° C. The method may also include performing analysis on the cell free RNA from within the sample.

The stabilizing blood collection device may include diazolidinyl urea and aurintricarboxylic acid. The stabilizing blood collection device may include aurintricarboxylic acid and sodium fluoride. The stabilizing blood collection device may include imidazolidinyl urea and glycine. The stabilizing blood collection device may include diazolidinyl urea and glycine. The stabilizing blood collection device may include diazolidinyl urea, aurintricarboxylic acid and sodium fluoride. The stabilizing blood collection device may include some combination of diazolidinyl urea, imidazolidinyl urea, aurintricarboxylic acid, glyceraldehyde, and sodium fluoride, and EDTA.

During the step of sample handling (which includes but is not limited to sample transport), the sample may be subjected to a temperature below room temperature (e.g., below about 15° C., 10° C., or even 7° C.) throughout at least a portion (e.g., at least one tenth, one quarter, or one half) of the duration of the handling step. During the step of sample handling the sample may be subjected to a temperature above room temperature (e.g., above about 25° C. or even 30° C.) throughout at least a portion (e.g., at least one tenth, one quarter, or one half) of the duration of the handling step. During the step of sample handling the sample may be subjected to irregular and/or uncontrolled periods of vibration and/or agitation. The duration of the handling step may be for at least about 24 hours. The duration of the handling step may be for at least about 72 hours. The step of performing cell free RNA analysis on the sample may include performing transcriptase real-time PCR (RT-qPCR). The step of performing cell free RNA analysis on the sample may include performing transcriptase real-time PCR (RT-qPCR) to quantify mRNAs for c-fos, β-actin, and/or 18S rRNA. The sample treated with the stabilizing agent may exhibit an increase in mRNA copy numbers that is less than 50% that of a sample handled in the absence of the treating step. The handling step may include transporting the sample from a blood draw site to a site for cell free RNA analysis in a transportation vehicle (e.g., selected from a truck, a train, an airplane, a helicopter, an automobile, a watercraft or the like).

The teachings herein further contemplate that the methods disclosed herein result in the cellular mRNA copy number per mL of plasma within the blood sample remaining substantially the same prior to shipping and post-shipping. The teachings herein further contemplate that the methods disclosed herein result in the c-fos mRNA copy number per mL of plasma within the blood sample remaining substantially the same prior to shipping and post-shipping. The β-Actin mRNA copy number per mL of plasma within the blood sample may remain substantially the same prior to shipping and post-shipping. The 18s rRNA copy number per mL of plasma within the blood sample may remain substantially the same prior to shipping and post-shipping. The amount of background RNA in the blood sample may remain substantially the same prior to shipping and post shipping. The c-fos mRNA copy number per mL of plasma within the blood sample may remain substantially the same when stored at 6° C., 22° C., and 30° C. for 3 days. The β-Actin mRNA copy number per mL of plasma within the blood sample may remain substantially the same when stored at 6° C., 22° C., and 30° C. for 3 days. The amount of background RNA in the blood sample may remain substantially the same when stored at 6° C., 22° C., and 30° C. for 3 days.

### BRIEF DESCRIPTION OF THE DRAWINGS

FIG. 1—Shipping temperature record—showing the temperature of selected blood samples over time during shipment.

FIG. 2A—C-fos mRNA copy number per milliliter of plasma—showing the mRNA copy number (using c-fos markers) in initial, not shipped and shipped blood samples contacted with EDTA and the same for blood samples located in the stabilizing blood collection devices taught herein.

FIG. 2B—β-Actin mRNA copy number per milliliter of plasma—showing the mRNA copy number (using β-Actin markers) in initial, not shipped and shipped blood samples contacted with EDTA and the same for blood samples located in the stabilizing blood collection devices taught herein.

FIG. 2C—18s rRNA copy number per milliliter of plasma—showing the rRNA copy number (using 18s rRNA markers) in initial, not shipped and shipped blood samples contacted with EDTA and the same for blood samples located in the stabilizing blood collection devices taught herein.

FIG. 3A—C-fos mRNA copy number per milliliter of plasma—showing the mRNA copy number (using C-fos mRNA markers) at initial draw, 6° C. storage temperature, 22° C. storage temperature and 30° C. storage temperature blood samples contacted with EDTA and the same for blood samples located in the stabilizing blood collection devices taught herein.

FIG. 3B—β-Actin mRNA copy number per milliliter of plasma—showing the mRNA copy number (using β-Actin mRNA markers) at initial draw, 6° C. storage temperature, 22° C. storage temperature and 30° C. storage temperature blood samples contacted with EDTA and the same for blood samples located in the stabilizing blood collection devices taught herein.

### DETAILED DESCRIPTION

This application claims the benefit of the priority date of U.S. Provisional Application Ser. No. 61/751,983, filed Jan. 14, 2013, the contents of which are incorporated by reference herein for all purposes. This application is also related to U.S. Pat. No. 8,304,187, filed on Feb. 11, 2010, the contents of which are incorporated by reference herein in its entirety.

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

Recent studies have shown blood plasma to contain low abundance cfRNA targets, an example of which is circulating tumor cell-derived RNA. Polymerase chain reaction detection of these cell-free targets within a high amount of cellular background RNA is challenging, requiring specialized protocols and/or large volumes of starting material. As a result, minimizing the release of cellular RNA from nucleated cells (e.g., background RNA) is essential for accurate analysis of true cfRNA.

In current practice, it is recommended that blood samples be immediately centrifuged to isolate and freeze plasma to prevent background RNA contamination of cfRNA during sample processing, transportation and storage. However, the examples discussed herein demonstrate that the stabilizing blood collection device of the present invention prevents the release of background RNA into plasma post-phlebotomy for up to 3 days, thus avoiding these labor-intensive requirements (e.g., centrifugation and freezing). Using the stabilizing blood collection device disclosed herein, ex vivo storage at room temperature becomes possible, allowing flexibility for offsite blood draws to be sent to central laboratories for downstream analysis of the cfRNA without preliminary centrifugations or cryopreservation.

As mentioned above, transportation of blood samples from the site of phlebotomy to another facility is commonly required for molecular diagnostic testing. In this regard, several studies have focused on pre-analytical variables that might compromise the accuracy cfRNA measurements, including the selection of blood collection devices, sample storage and shipping conditions. Each of these parameters affects the amount of nucleated blood cell lysis that occurs post-phlebotomy. Such nucleated cell lysis leads to release of cellular RNA, elevating RNA backgrounds and suppressing true and accurate cfRNA measurement. Accordingly, inaccurate cfRNA measurement reduces or even eliminates the utility of such measurements for disease diagnosis, genetic testing or other purposes. The devices disclosed herein allow for minimized background RNA increases by utilizing a stabilizing blood collection device with the ability to stabilize nucleated blood cells during shipping movement and temperature fluctuations. The examples discussed herein evaluate the ability of the stabilizing blood collection device taught herein and traditional K₃EDTA tubes to preserve cfRNA and prevent background RNA release when subjected to conditions that can occur during sample storage and shipping.

During transport of samples, shaking may disrupt nucleated blood cell integrity and compromise accuracy of sample testing, as described above. For the examples described herein, blood samples were shipped in either K₃EDTA tubes or the stabilizing blood collection device taught herein. The resulting mRNA or rRNA copy numbers (as measured by c-fos, β-Actin or 18s RNA markers) showed increased background RNA for the shipped samples in the K₃EDTA tubes as compared to those samples in the stabilizing blood collection device taught herein. The stabilizing blood collection device samples showed stable mRNA or rRNA copy numbers before and after shipping. This suggests nucleated cell disruption occurred in blood samples that were shipped in K₃EDTA leading to cellular RNA release, as this did not occur in the samples within the stabilizing blood collection device.

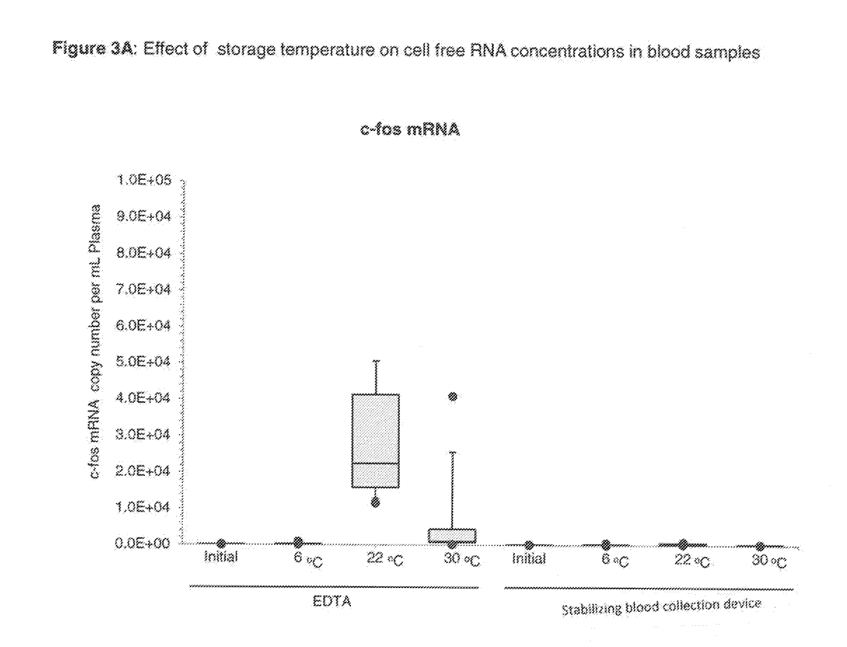

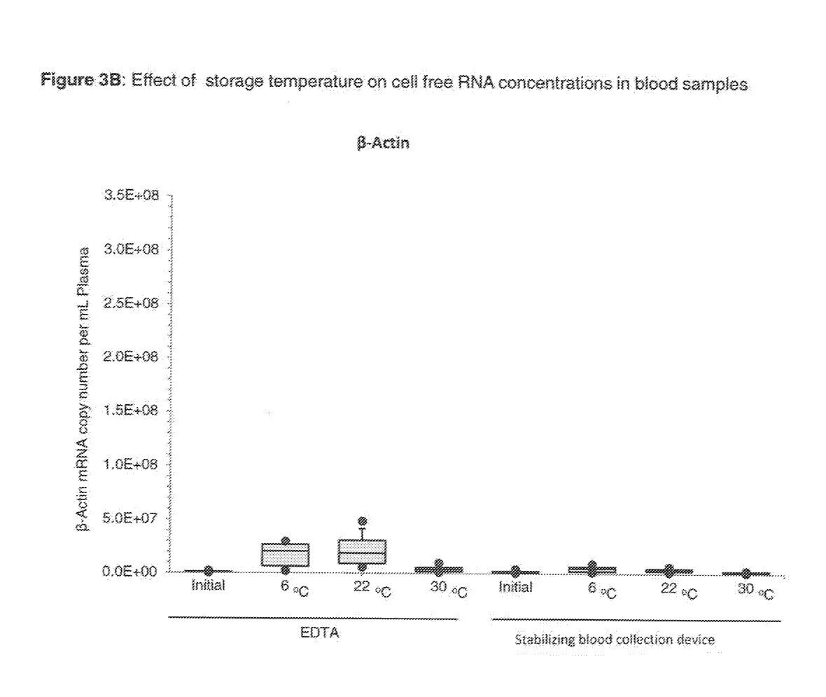

Variation in sample storage temperature is another post-phlebotomy condition that can cause undesirable changes in background RNA concentration. Here, we studied the effect of three different storage temperatures on the mRNA copy numbers of blood drawn into K₃EDTA and the stabilizing blood collection device taught herein. Following blood draw, samples were incubated at 6° C., 22° C. or 30° C. for 3 days. Significant increases in K₃EDTA blood sample background RNA concentrations were seen at 6°, 22° C. and 30° C. by day 3 (as measured by c-fos, and β-Actin markers) (FIGS. 3A-3B). As shown, blood drawn into the stabilizing blood collection device showed no significant increase in mRNA copy number at any temperature on day 3.

The stabilizing blood collection device may include diazolidinyl urea and aurintricarboxylic acid. The stabilizing blood collection device may include aurintricarboxylic acid and sodium fluoride. The stabilizing blood collection device may include imidazolidinyl urea and glycine. The stabilizing blood collection device may include diazolidinyl urea and glycine. The stabilizing blood collection device may include diazolidinyl urea, aurintricarboxylic acid and sodium fluoride. The stabilizing blood collection device may include some combination of diazolidinyl urea, imidazolidinyl urea, aurintricarboxylic acid, glyceraldehyde, and sodium fluoride, and EDTA. The imidazolidinyl urea may be present in an amount of from about 100 g/l to about 1000 g/l. The imidazolidinyl urea may be present in an amount of from about 300 g/l to about 600 g/l. The diazolidinyl urea may be present in an amount of from about 50 g/l to about 800 g/l. The diazolidinyl urea may be present in an amount of from about 100 g/l to about 400 g/l. The EDTA may be present in an amount of from about 20 g/l to about 150 g/l. The EDTA may be present in an amount of from about 50 g/l to about 100 g/l. The glycine may be present in an amount of from about 10 g/l to about 150 g/l. The glycine may be present in an amount of from about 35 g/l to about 100 g/l. The glyceraldehyde may be present in an amount of from about 10 g/l to about 150 g/l. The glyceraldehyde may be present in an amount of from about 35 g/l to about 100 g/l. The aurintricarboxylic acid may be present in an amount of from about 1 g/l to about 40 g/l. The aurintricarboxylic acid may be present in an amount of from about 5 g/l to about 20 g/l. The sodium fluoride may be present in an amount of from about 0.1 g/l to about 30 g/l. The sodium fluoride may be present in an amount of from about 0.5 g/l to about 10 g/l.

For the examples below, blood donors were recruited with informed consent from Streck, Inc. in Omaha, Nebr. Donors were both male and female and presumed to be healthy. All draws were performed using venipuncture.

### EXAMPLES

To study the effect of storage temperature on cfRNA concentration, samples were stored at 6°, 22° and 30° C. for 3 days. For each experiment, plasma was separated at various time points by centrifugation at 300×g for 20 min followed by transferring the upper plasma layer to a new tube, and then re-centrifuging at 5,000×g for 10 min. Total plasma cfRNA was extracted and reverse transcriptase real-time PCR (RT-qPCR) was used to quantify mRNAs for c-fos, β-actin, and 18S rRNA.

To study the effect of transportation, blood was drawn from 10 donors. Blood was drawn from each donor into three 10 mL K₃EDTA tubes and three 10 mL stabilizing blood collection devices in accordance with the teachings herein. One K₃EDTA tube and one stabilizing blood collection device from each donor were processed within two hours (2 h) of blood draw. Another K₃EDTA tube and stabilizing blood collection device from each donor were shipped with a temperature tracking device to a laboratory in Springfield, Mass. and back to Nebraska during the course of three days. The remaining K₃EDTA tube and stabilizing blood collection device from each donor were kept at 22° C. for three days and processed with the returned shipped blood tubes.

### RESULTS

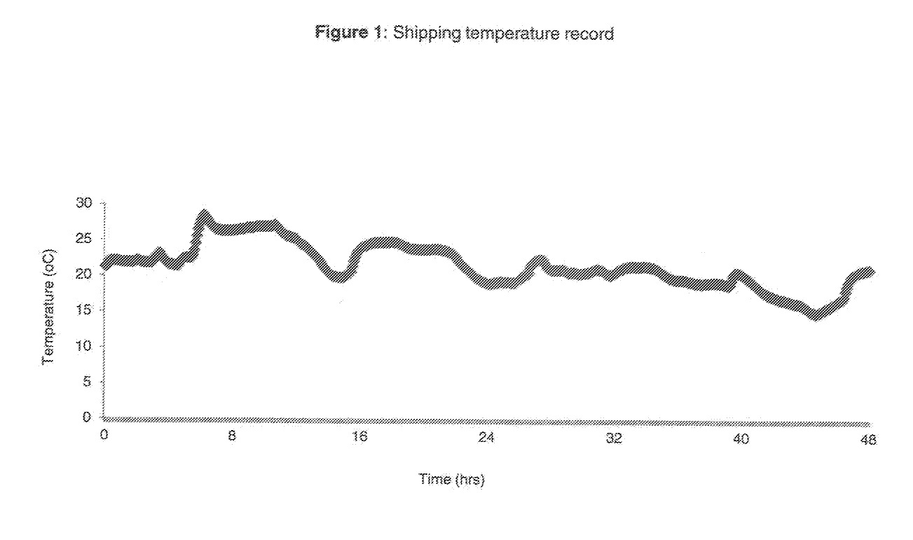

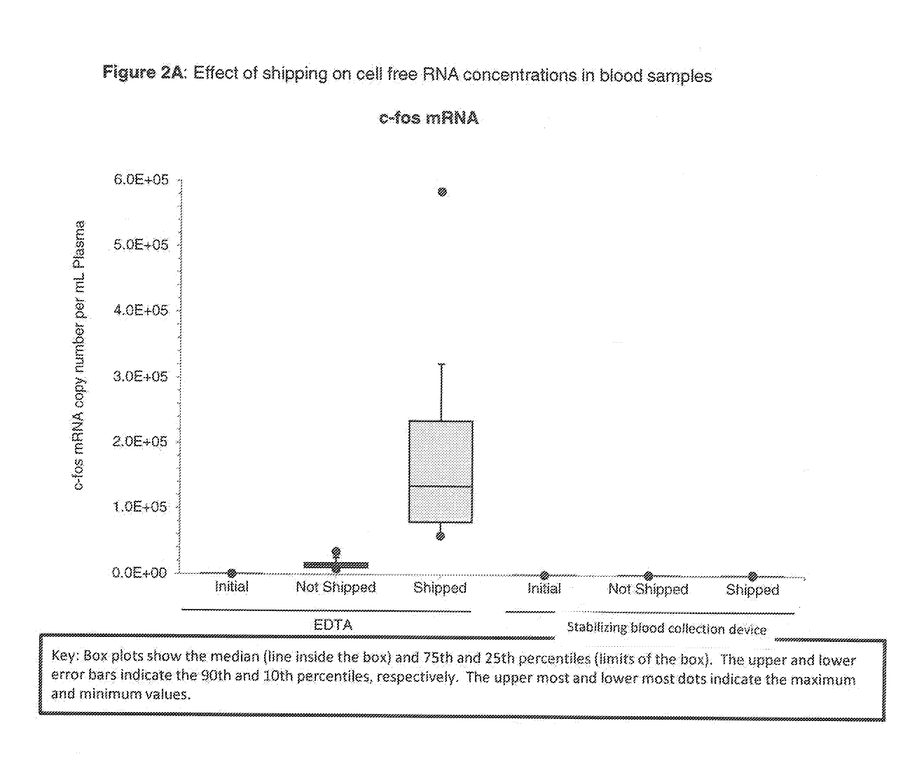

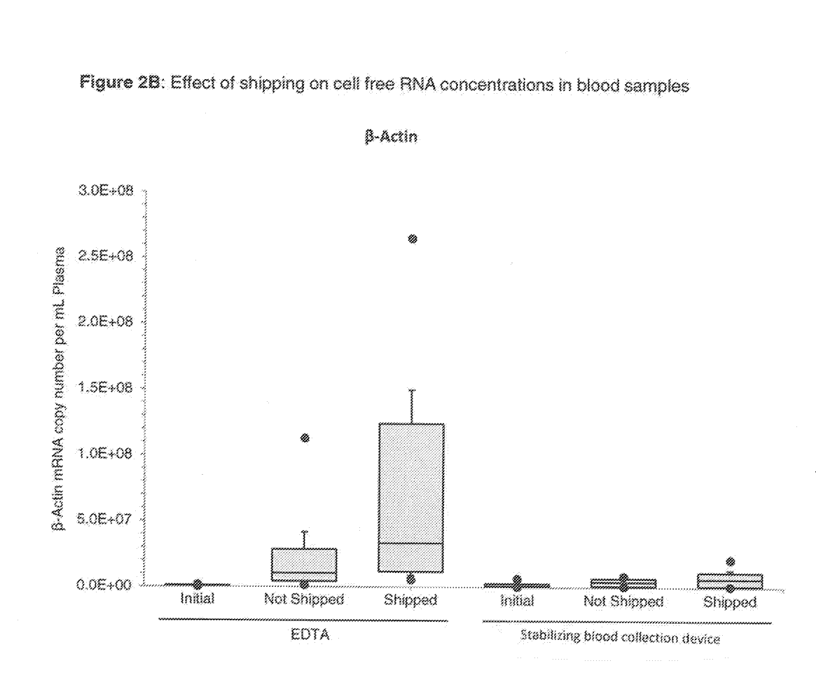

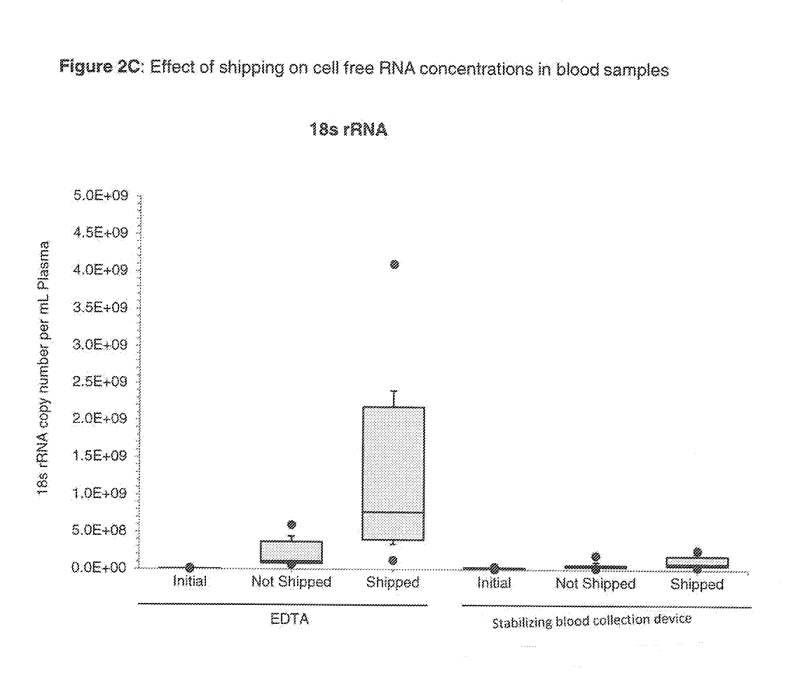

The temperature tracking device kept inside the shipping container showed a temperature range of 15°-29° C. (see FIG. 1), As demonstrated at FIGS. 2A-2C, shipped blood samples drawn into K₃EDTA tubes showed a significant increase in mRNA or rRNA copy numbers for β-actin, c-fos, and 18S rRNA. In contrast, shipped blood samples drawn into stabilizing blood collection devices showed only a slight change in mRNA or rRNA copy numbers for β-actin, c-fos, and 18S rRNA. As shown in FIGS. 3A-3B, blood stored in K₃EDTA tubes at 6, 22 and 30° C. for 3 days showed a significant increase in mRNA copy numbers for β-actin or c-fos. Compared to K₃EDTA tubes, blood stored in stabilizing blood collection devices at 6, 22 and 30° C. for 3 days showed slight to moderate increases in mRNA copy numbers for β-actin and c-fos.

The results above demonstrate that the stabilizing blood collection devices taught herein have a dramatic and previously unrecognized effect on the amount of background RNA that results from shipping and storage temperatures above freezing. It can be appreciated that the stabilizing blood collection devices not only protect target nucleic acids from degradation, but also maintain the quantity of such target nucleic acids. As a result, these quantities remain substantially stable without the intrusion of background nucleic acids so that the relative quantities can be effectively utilized for measurements that may assist in disease diagnosis and tracking.

It will be appreciated that concentrates or dilutions of the amounts recited herein may be employed. In general, the relative proportions of the ingredients recited will remain the same. Thus, by way of example, if the teachings call for 30 parts by weight of a Component A, and 10 parts by weight of a Component B, the skilled artisan will recognize that such teachings also constitute a teaching of the use of Component A and Component B in a relative ratio of 3:1. Teachings of concentrations in the examples may be varied within about 25% (or higher) of the stated values and similar results are expected. Moreover, such compositions of the examples may be employed successfully in the present methods to isolate fetal nucleic acids (e.g., cell-free RNA).

It will also be appreciated that the above is by way of illustration only. Other ingredients may be employed in any of the compositions disclosed herein, as desired, to achieve the desired resulting characteristics. Examples of other ingredients that may be employed include antibiotics, anesthetics, antihistamines, preservatives, surfactants, antioxidants, unconjugated bile acids, mold inhibitors, nucleic acids, pH adjusters, osmolarity adjusters, or any combination thereof.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

1. A method for handling a biological sample to a remote site or cell-free RNA analysis, comprising the steps of:
a. drawing a blood sample directly into a stabilizing blood collection device at a blood draw site, such blood collection device including an effective amount of a stabilizing agent selected from imidazolidinyl urea, diazolidinyl urea, aurintricarboxylic acid, glycine, glyceraldehyde, sodium fluoride or combinations thereof, the sample of blood including an initial amount of background RNA and an amount of cell free RNA;
b. treating one or more components of the sample with the stabilizing agent for mitigating the propensity of background RNA within the sample to increase relative to the initial amount;
c. handling the sample, while it remains in the blood collection device, for delivery to site at which cell-free RNA analysis is to be performed, during which transporting step the blood collection device and the sample contained therein is subjected to one or more temperatures within a range of about 5 to about 35 ° C.;
d. performing cell free RNA analysis on the sample

2. The method of claim 1, wherein during the step of sample handling the sample is subjected to a temperature below room temperature (e.g., below about 15° C., 10° C., or even 7° C.) throughout at least a portion (e.g., at least one tenth, one quarter, or one half) of the duration of the handling step.

3. The method of claim 1, wherein during the step of sample handling the sample is subjected to a temperature above room temperature (e.g., above about 25° C. or even 30° C.) throughout at least a portion (e.g., at least one tenth, one quarter, or one half) of the duration of the handling step.

4. The method of claim 1, wherein during the step of sample handling the sample is subjected to irregular and/or uncontrolled periods of vibration and/or agitation.

5. The method of claim 1, wherein the duration of the handling step is for at least about 24 hours.

6. The method of claim 1, wherein the duration of the handling step is for at least about 72 hours.

7. The method of claim 1, wherein the step of performing cell free RNA analysis on the sample includes performing transcriptase real-time PCR (RT-qPCR).

8. The method of claim 1, wherein the step of performing cell free RNA analysis on the sample includes performing transcriptase real-time PCR (RT-qPCR) to quantify mRNAs for c-fos, β-actin, and/or 18S rRNA

9. The method of claim 7, wherein the sample treated with the stabilizing agent exhibits an increase in mRNA copy numbers that is less than 50% that of a sample handled in the absence of the treating step (b).

10. The method of claim 1, wherein the handling step includes transporting the sample from a blood draw site to a site for cell free RNA analysis in a transportation vehicle (e.g., selected from a truck, a train, an airplane, a helicopter, an automobile, a watercraft or the like).

11. The method of claim 1, wherein the c-fos mRNA copy number per mL of plasma within the blood sample remains substantially the same prior to shipping and post-shipping.

12. The method of claim 1, wherein the β-Actin mRNA copy number per mL of plasma within the blood sample remains substantially the same prior to shipping and post-shipping.

13. The method of claim 1, wherein the 18s rRNA copy number per mL of plasma within the blood sample remains substantially the same prior to shipping and post-shipping.

14. The method of claim 1, wherein the amount of background RNA in the blood sample remains substantially the same prior to shipping and post shipping,

15. The method of claim 1, wherein the c-fos mRNA copy number per mL of plasma within the blood sample remains substantially the same when stored at 6° C., 22° C., and 30° C. for 3 days.

16. The method of claim 1, wherein the β-Actin mRNA copy number per mL of plasma within the blood sample remains substantially the same when stored at 6° C., 22° C., and 30° C. for 3 days.

17. The method of claim 1, wherein the amount of background RNA in the blood sample remains substantially the same when stored at 6° C., 22° C., and 30° C. for 3 days.
